(19) United States
(12) Patent Application Publication
(10) Pub. No.: US 2007/0039241 A1
(43) Pub. Date: Feb. 22, 2007

(54) Floatable granular substrate for culturing plant material

(75) Inventors: Florian Battke, Unterhaching (DE); Ernst Dietrich, Gauting (DE)

Correspondence Address: MONAHAN & MOSES, LLC, 13-B W. WASHINGTON ST., GREENVILLE, SC, 29601, US

(73) Assignee: GSF - Forschungszentrum fur Umwelt und Gesundheit, Oberschleissheim (DE)

(21) Appl. No.: 10/577,416

(22) PCT Filed: Oct. 27, 2004
(86) PCT No.: PCT/EP04/12144

§ 371 Date: Jun. 8, 2006

(30) Foreign Application Priority Data
Oct. 27, 2003 (GB) 0325019.8

(51) Int. Cl. A01G 31/06; A01G 31/00

(52) U.S. Cl. 47/59 S; 47/59 R

(57) ABSTRACT

The present invention provides a method of culturing plant material comprising a layer of floatable granular substrate in a culturing vessel, plant material and culture medium. Also, the invention provides a culturing kit comprising various combinations of floatable granular substrate with plant material, culturing solution and a culturing vessel, adapted to the specific requirements of hobby, science or industrial uses.

### TECHNICAL FIELD OF THE INVENTION

The invention relates to a method for culturing plant material which makes use of a floatable granular substrate, and a culturing kit which comprises said substrate together with a culturing vessel.

### BACKGROUND OF THE INVENTION

The use of hydroponic systems for culturing plant material including whole plants, seeds, seedlings, meristems or calluses is widespread in science and industry. In particular, biotechnological methods of plant propagation, modification or culture typically involve hydroponic systems at least at some stage. Such biotechnological methods are typically used in science, but also are in routine use in generating plant material for industrial purposes, both in agriculture and the production of ornamental flowers or plants.

Hydroponic systems are often chosen instead of conventional soil in order to provide chemically defined nutrients to plants. As a matter of fact, this is a technical necessity for many biotechnological propagation methods where defined cocktails of phytohormones need to be administered at certain points in the development of the plant. Furthermore, nutrient solutions are often easier to handle and to sterilize as compared to soil.

In scientific settings, hydroponic systems are often used in combination with phytotronic cabinets and exposure chambers supplying defined artificial light quality, quantity, direction and temporal variation (Thiel et al, 1996). Together, these measures allow to create reproducible and defined growth conditions for the experiments.

Apart from growing plants under standardized nutrient supply, hydroponic systems also allow simple addition of substances to the nutrient solution, without any such interferences that could be expected for plants cultured in soil. Further, plant material such as roots can readily be isolated from hydroponic systems for further analysis.

In hydroponic systems, plants are typically grown directly in granulated substrates such as expanded clay. Culture substrate is provided for mechanical support and an appropriate tactile environment for root growth. Alternatively, in particular for scientific applications, highly specialized vessels, rockwool- or agar-based systems can be used (Gibeaut et al, 1996; Heidenreich, 1999; Hattner and Bar-Zvi, 2003; Tocquin et al., 2003).

However, such systems have considerable limitations:

In conventional hydroponic systems using granulated substrate, the level of the nutrient solution needs to be carefully adjusted to an optimal height in relation to the substrate to provide optimal culturing conditions. If the nutrient solution is too high, the plant material may encounter detrimental anaerobic conditions. On the other hand, if the level of nutrient solution is too low, the plant material may fall dry. This is of particular relevance for immature plant material, especially such without an established root system.

Also, granulated substrates often contain clefts that are too large for growing plants from small seeds, as these may easily fall into a cleft where they do not find optimal growth conditions. Therefore, seedlings are often germinated on one particular substrate (e.g. filter paper) and are then transferred into hydroponic culture. Such transferral easily results in mechanical stress for the seedlings and furthermore is very labour and time intensive.

One improvement over hydroponic systems based on granulated substrates are floating culturing systems. Such floating systems can provide optimal growth conditions for differing levels of nutrient solution in the culturing container. One example of a floating system used in agriculture e.g. for the cultivation of tobacco seedlings contains a polystyrene-float with a number of openings, in which seedlings can be placed (Leal, 2001). However, each individual plant needs to be placed manually into one of the openings, hence this method is very labour intensive. Also, the seedling is supported in the opening by either soil, or a substrate like e.g. rockwool. These substrates are unsuitable for a number of applications.

In U.S. Pat. No. 4,916,856 (and closely related FR 2590443, EP0156749, and FR2673072)automated plant culturing installations are disclosed, which employ granulates such as expanded clay, vermiculit, perlite, glass, etc. These automated plant culturing installations comprise complex mechanical devices for the automated moving of the plants from the centre of a circular vat to its periphery. For example, guide wire systems, both embedded in the granulate and above are disclosed.

Any system employing soil or other conventional substrates, such as expanded clay or other minerals, is limited by the fact that such substrate is not chemically inert. In particular, such substrates can readily adsorb components of the nutrient solution. This is a considerable disadvantage when the nutrient solution needs to contain a defined concentration of a specific ingredient. Such is the case e.g. in many scientific experiments, or in biotechnological culturing methods wherein defined concentrations of phytohormones must be present at given growth periods.

Further to causing variation of concentrations of a desired ingredient, such adsorption to the substrate may necessitate the addition of larger quantities of the ingredient to obtain the desired final concentration. In case of expensive ingredients, such as many phytohormones, this can represent a substantial economic disadvantage, in particular for biotechnological methods of cultivation.

To overcome the limitations resulting from the adsorption of chemicals to the substrate, agar-based systems can be employed. However, these are expensive and laborious to set up. Furthermore, once the roots of the plant grow into the agar, the substrate cannot readily be exchanged. Hence, the temporally controlled addition or withdrawal of substances is limited. A further limitation of agar based systems is the adherence of the substrate to root material. When such a plant is replanted into e.g. soil for further growth, the agar residues are prone to bacterial degradation. This process of bacterial agar decomposition can result in unfavourable conditions for the plants. Further, residual agar attached to the plant material poses a,-technological limitation to e.g. experiments that study the uptake of certain chemicals by the plant and require the analysis of plant material absolutely free of surrounding substrate.

Alternative culture systems have been described. For Arabidopsis thaliana a floating sponge system for growth of individual plants has been used (Arteca & Arteca, 2000). Aluminium tolerance of barley was tested with a 50 ml syringe system, which allowed the growth of 5 seedlings (Feng et al., 1997). However, both systems are laborious to handle, and cannot readily be scaled up. Hence, they cannot be used for industrial application or scientific experiments requiring large numbers of individual plants.

Osmotek Ltd., Rehovot, Israel, commercialises a floating culturing system (www.osmotek.com/product.htm#LifeRaft and www.osmotek.com/liferaftDescription.html). In this system, a semipermeable membrane is placed inside a frame. This frame is then supported by a float that allows the membrane to be in contact with the nutrient solution. Float and frame are placed inside a proprietary culturing container filled with nutrient solution.

In particular for larger plant material this system is very laborious, as the plants must be placed in so called support sockets to prevent them from falling over, thereby losing good contact between the culture material base and the membrane.

The semipermeable membrane does not allow root penetration. Hence, there is no direct contact of plant and nutrient solution. This may affect both nutrient exchange and accumulation of toxic exudates. Alternatively, however, membranes with holes are available, that allow root growth into the nutrient solution.

However, if plant material is growing through holes in the membrane, it is very difficult to isolate seedlings from the membrane without destruction of root tissue. Also, such membranes cannot readily be reused.

Even if the membranes have holes, contact of plant material with the nutrient solution is restricted by the float. Though the float contains a central opening to allow solution exchange, all regions not directly apposing this opening only enjoy a very thin solution support of a few millimetres. The practical consequence of limited or missing contact with a large solution phase is a limited nutrient supply on the one hand, and accumulation of toxic plant exudates on the other hand.

This culturing system brings about a number of further limitations. For example, the user is restricted to specialized culturing containers, that firstly are expensive, and secondly cannot readily be scaled up, as the integrated system is only available in few defined sizes. Furthermore, any float is laid out to support a defined plant weight. If the plant weight differs from that weight, it can become necessary to change the float.

A further limitation of the membrane system is that primary roots may not receive the optimal tactile signals necessary for development. This is of particular importance, if the plant material is meant to be replanted for further cultivation, or if entire plants of normal morphology are needed for scientific purposes.

Accordingly, there is need for a culturing system that overcomes all of these limitations. In particular, the culturing system should readily be scaleable from laboratory to industrial scale, should be easy to set up without laborious preparation, should be made of cheap components and allow use of various culturing containers. Additionally the system should allow culturing of different plant materials, including seeds of different sizes, seedlings, plants, meristems, calluses or other cellular aggregates. In particular, the culturing system should allow culturing of plant material bearing roots, and should allow easy harvesting and replanting of such material, it should provide plant material with sufficient nutrients and dissipate toxic exudates over a prolonged period of time, should allow easy exchange of culture medium, it should be chemically inert not to adsorb ingredients of the culture medium and should be sterilizable by conventional means.

### SUMMARY OF THE INVENTION

The present invention concerns a culturing method for plant material, wherein a floatable culture substrate, a culture medium and plant material supported by the culture substrate are in a self-regulated balance, such that the culture medium maintains an optimal height in the culture substrate throughout the culturing process, irrespective of changes in the height of culture medium in the culturing container (provided there is enough medium that the substrate can float) and/or plant growth. Thus, the characteristics of the granular substrate layer (material, thickness of the layer etc.), the culturing medium (density, etc.) and the plant material (size, weight, shape, etc.) are selected such that there exists said balance in a floating state.

The present invention provides the use of a floatable granular substrate for culturing plant material, wherein said floatable granular substrate comprises particles having an average diameter in the range of 1-25 mm, more preferred 1-10 mm, a regular or irregular spheroid or polygonal shape and a smooth surface.

The granular substrate of the invention forms a substrate layer, wherein said layer may comprise one type of granules (e.g. made of one material), or may comprise different types of granules. Said different types of granules may differ e.g. with respect to their material, size, coating, etc.

Another preferred embodiment comprises a granular substrate that is chemically inert.

Another preferred embodiment comprises a granular substrate with a density of 50-99.8% of the density of the culture medium used, or a density in the range of 0.5-1.1 g/cm3.

Another preferred embodiment comprises a granular substrate being a thermoplastic polymer. Said thermoplastic polymer can be any one selected from high density polyethylene (HD-PE), low density polyethylene (LD-PE) or polypropylene (PP), wherein individual granules may comprise a single one of said polymers, or a mixture thereof. Also, the granule layer may comprise a multitude of individual granules, which differ from one another, e.g. with respect to the material they are made of.

Thus, another preferred embodiment comprises composite particles composed of more than one component, wherein said components can individually be more or less dense than the average density of the particle, and/or particles comprising at least one hollow enclosure.

Another preferred embodiment comprises a granular substrate sterilizable by a chemical treatment, irradiation and/or heat.

In one preferred embodiment, the granular substrate is forming a floatable substrate layer with a height of 0.5-20 cm, and preferably 0.5-10 cm that may or may not float on a culture medium which may or may not be aerated.

In another embodiment the granular substrate layer comprises embedded additional support structures.

According to a further aspect, the present invention provides a culturing kit for culturing plant material comprising a floatable granular culture substrate. Said substrate can be combined with any one, two or all of the components culturing solution, plant material and culturing vessel. Granular substrates of all previously specified embodiments can be used for said kit.

According to a further aspect, the present invention provides a method for culturing plant material comprising:

(a) forming a layer of floatable granular substrate in a culturing vessel,

(b) placing plant material on or in said layer, and

(c) culturing the plant material in the presence of a culture medium, wherein there is no additional structure supporting the plant material from underneath, and wherein the culture medium is added before or after the layer of the granular substrate is formed.

In a preferred embodiment, there may be additional support structures present, provided they do not support the plant material from underneath. Preferably, the additional support structure is embedded in and supported by the culturing substrate. In a preferred embodiment, such additional support structure is not fixed. Fixed in this context means, that if e.g. the level of medium in the culturing container changes, resulting in a corresponding movement of the floatable granular substrate, the fixed support structure shows no corresponding movement. Thus, for a fixed support structure the relative position of the support structure and the granular substrate to each other change depending on the medium level.

The method can be performed using any of the previously specified culturing kits or any of the previously specified embodiments of the granular substrate.

### BRIEF DESCRIPTION OF THE FIGURES

FIG. 1: Cultivation of barley seedlings on a floating layer of LD-PE substrate.

FIG. 2: Representative example of (a) six day old barley seedling harvested from LD-PE substrate, and (b) tobacco derived meristems showing rich primary root development harvested from PP substrate after 10 days of culturing.

FIG. 3: Example of calluses cultivated on floating LD-PE substrate (a) a lateral view, and (b) a top view.

FIG. 4: Example of meristems derived from tobacco on floating PP substrate in a glass culturing vessel. (a) a lateral view and (b) a top view.

### DETAILED DESCRIPTION OF THE INVENTION

Substrate Material

Conventional substrates such as expanded clay, rockwool, soil or agar are not floatable. If floating is desired, such substrates must be suspended by a separate floating entity. In contrast, the present invention provides a floatable granular substrate for culturing plant material.

The material for the substrate according to the present invention is not limited, as long as this material is floatable in a culturing solution, and provides mechanical stability and tactile stimuli for root growth in a hydroponic system. The elasticity of the substrate material is not limited. Hence, any materials ranging from highly stiff to highly elastic can be used. Preferably, the material is chemically inert. Examples of substrates with these characteristics comprise, but are not limited to, thermoplastic polymers of appropriate density. Specific examples of such thermoplasts comprise polypropylene (PP), high density polyethylene (HD-PE) or low density polyethylene (LD-PE). These polymers are used in large quantities in a wide range of industrial applications and are typically provided in the form of a raw material granulate. Therefore, such granulate can readily be obtained at low cost and in any reasonably desired quantity. The granulate can be directly used as a culture substrate, or be further processed by e.g. selecting a certain size fraction, sterilization, packaging, etc.

For particular applications, a preferable granulate material may be degradable in a defined way. Examples of such materials comprise, but are not limited to e.g. extruded starch polymers. It may be required to coat such degradable materials in an appropriate fashion to achieve the desired properties. It is obvious to the skilled person, what kind of degradable composition to use for a given application.

In the present specification, the term “culturing” of plant material is used in its widest sense. This means that plant material is generally kept under such conditions which the skilled person expects the plant material will survive at. However, that does not exclude that under certain circumstances conditions will deliberately be chosen such that the plant material does not survive. Culturing can, but need not, mean that plant cells divide or differentiate. Examples of such processes include growing seedlings from seeds, differentiating calluses, or cultivating meristems.

In the context of the present invention, the terms “plant” and “plant material” are used in their widest senses and are meant to include all conceivable elements derivable from a plant, such as organs (e.g. roots, leaves, etc), tissue isolated thereof (e.g. meristems), or individual cells or cellular aggregates (e.g. calluses) isolated thereof. It further comprises all stages of plant development, comprising but not limited to seeds, seedlings or fully grown plants.

The term “granular” means that the substrate is composed of a multitude of individual particles. Said particles are not firmly attached to one another. Individual particles can be of differing size or shape, within a certain degree of freedom, such that a given culture substrate comprises a multitude of similar particles.

For the present invention, the precise shape of the granules is not limited, but will preferably approximate to a spheroidal shape, or a polygonal shape. Other examples of possible shapes comprise, but are not limited to regular or irregular polygonal shapes, cubes, octahedra, tetrahedra, pyramids etc. In this context, “spheroidal” means that the particles of the granulate have a globe or ball shape in the broadest sense. This does not preclude that individual particles or the majority of the particles of the granulate approximate the shape of an ideal sphere.

Typically, however, the particles of the predominant shape will deviate from the ideal geometrical shape and bear some kind of irregularity. This means e.g. that particles can be ball shaped, ellipsoid, pear shaped or any other shape that in the widest sense can be considered spheroidal. Furthermore, the particles can contain clefts, ridges, corners or edges, one or a few spikes or one or a few holes.

In other words, the overall appearance of the particles preferably resembles spheres or polyhedrons to some degree. This is a requirement to form floatable layers of sufficient stability to carry the weight of plant material. The irregularity of shapes and the size distribution in the granulate will have an impact on packing density of the substrate. The more individual particles approximate ideal spheres and the more homogenous the size distribution, the more the packing will approximate the theoretical optimum of tightly packed spheres.

Though the shape of individual particles can be irregular, the surface of the particles preferably is smooth. This does not preclude the presence of one or a few clefts, ridges, corners or edges, one or a few spikes or one or a few holes. In particular, the substrate preferably does not have any extensive surface protuberances or cavities that comprise a separate solution phase

A typical example of a granular substrate comprising such a separate solution phase is expanded clay. The expanded clay particles have many holes and cavities forming extended inner volumes and surfaces. This has the disadvantage, that the liquid phase in these inner cavities can have different characteristics as compared to the surrounding culturing medium, e.g. after exchange of medium for a chemically different medium. A slow exchange of the two media would then ensue, wherein the medium is leaching from the cavities into the main body of medium, and thereby contaminates that main body medium. Thus, a temporally defined exchange of chemically defined media is not possible in substrates such as expanded clay.

Hence, the substrate of the invention preferably does not contain extensive inner surfaces and cavities that communicate with the outside. One example of a substrate according to the invention is such that comprises no holes or pores at all. However, one or a few such pores and cavities may be present, in particular if the diameter of their opening to the surrounding solution is relatively large in relation to their volume. An alternative example of a particle of the present invention is made of a material that comprises inner cavities that do not communicate with the surrounding medium, i.e. a particle with a closed outer surface. Alternatively, the material of the particle may be repellent to the surrounding medium, such that even in the presence of openings, the medium would not enter the inner cavities.

The colour and transparency of the particles is not limited in the present invention. It can range from transparent to opaque and from black to white, including any chosen colour. For specific applications, transparent particles will be preferred, or such particles that have defined optical properties. Transparent particles may allow the optical investigation of the rhizosphere. If light of certain wavelengths, e.g. laser light, or fluorescence of certain wavelengths should be used in such investigations, the granulate must have suitable optical properties that can readily be chosen by the skilled person.

The average diameter of the individual granules is 1-25 mm. Such a size facilitates formation of a floatable substrate layer of adequate stability. In a preferred application, the size- and shape-distribution of commercially available industry grade polymer granulate, such as PE or PP granulate, is suitable to form a substrate layer according to the invention. Granulate that contains irregular, round shapes with an average diameter ranging from 1-8 mm is preferably used. For specific uses, particle sizes representing a fraction of this range, e.g. 1-3 mm or 2-5 mm may be particularly preferred.

The granular structure of the substrate used in the present invention will result in the formation of clefts in the substrate layer, which will be filled with nutrient solution. The degree of cleft filling depends on capillary effects that are influenced by the size and shape of the granules. Further, cleft filling is influenced by the relative density of the substrate in relation to the nutrient solution.

The density of the granular substrate must be such that it allows the substrate to float on standard nutrient solutions (nutrient solution, culturing solution and medium are used synonymously). Thus, the density of the substrate will be lower than that of the solution. At the same time, it must not be so low that the substrate layer sits on top of the solution in such a way that the clefts between the substrate granules are not filled with solution. Therefore, an optimum relative substrate density in relation to solution density exists. This optimum is within the range of 99.8 to 50% of solution density, preferably 80 to 99.8%, more preferably 90 to 99.8%, most preferably 95 to 99.8%.

In case the granulate comprises a composition of more than one substance, the mean density of the final granulate as used needs to fall within this range. Hence, single components of such a composite granule may have densities that do not fall within the specified ranges. For example, it can be envisaged that individual particles of the substrate may comprise gas filled enclosures, such as materials with a closed foam structure. In this case, the particle may comprise wall material with a density that may by far exceed the density of the culture medium. Alternatively, particles may be comprised in part by high density material and in part by low density material. Materials that may be used for composite particles comprise, but are not limited to, polyvinylchloride (PVC)., polymethylmetacrylate (PMMA), polytetrafluorethylene (PTFE), polycarbonate (PC), polyisoprene (PI), polyamide (PA), polyisobutylene (PIB), polyurethane (PU) and polystyrene (PS).

For a preferred application, it is important that there is no absence of solution filling in a significant upper segment (e.g. a quarter of the substrate layer) of the layer. Also, the substrate layer in a preferred application is not immersed such as to be covered by a closed layer of solution, as it is desired to grow plant material positioned at the interface of solution and air.

However; for specific applications, such as studying the effect of draught or flooding, the density of the culture substrate may be chosen such that either a significant layer of substrate devoid of culture solution exists, or the substrate is fully immersed.

Many culturing solutions have a density approximating 1 g/cm³. Specific examples of such solutions are e.g. Hoagland's E-Medium, ASTM STP 1027, or MS-basal medium (available from Sigma, M5519). For such nutrient solutions, preferably the density of the floatable granular substrate will be in the range of 0.5-1.00 g/cm³. A preferred density range is between 0.90-0.96 g/cm³. Specific examples of granular substrate in the desired density range are high density polyethylene (PE-HD, 0.94-0.96 g/cm³), low density polyethylene (PE-LD, 0.914-0.928 g/cm³), or polypropylene (PP, 0.90 g/cm³).

However, for special applications where nutrient solutions of higher density are used, such as e.g. studying plant growth under high salt concentrations, the density of the culturing substrate may be in excess of 1 g/cm³, e.g. 1.1 g/cm³.

Hence, the absolute density ranges of the substrate specified are not limiting. Rather, under any specific condition chosen, the optimum proportion of densities of solution and substrate must be conserved to ensure the culturing substrate is floatable on the specific solution used.

It is important in the context of the present invention that the floatable culturing substrate can provide such support from the beginning of the culturing process to the chosen plant material, and in particular small plant material such as certain seeds, that the plant material is prevented from sinking too deeply into the culture solution. In other words, the culturing substrate, the plant material and the medium must be in a self regulated balance.

Hence, any substrate that requires additional mechanical devices at least at some point during the culturing process that support the plant material from below to prevent its sinking, is not a culturing substrate in the sense of the present invention. For example, this applies when plant material is supported by a horizontal structure that allows plant access to nutrients like a mesh, membrane or filter, wherein the plant material is fixed onto this horizontal structure by adding some kind of substrate from above. Such a covering layer is not a culturing substrate in the sense of the present invention, as it is prevented from being in a balanced state by the fixed support structure.

An additional feature that is desirable for some applications is that the substrate can readily be sterilized by conventional means, such as irradiation (gamma-irradiation, UV, etc.), chemical treatment, autoclaving or any combination thereof. For example, the substrate can be sterilized by a chemical treatment using any conventional chemical sterilizing agent. Such agents can contain e.g. halogens or halogenated compounds (Cl, I, Br, F), lower alcohols (e.g. ethanol, propanol), phenols or phenol derivatives, aldehydes (e.g. formaldehyde, glutaraldehyde), quaternary nitrogen compounds, amphoterics, compounds liberating reactive oxygen species (e.g. H₂O₂), NaOCl, or ethyleneoxide. Alternatively, the substrate can be gamma-irradiated. Alternatively, the substrate can be autoclaved at conditions known to the skilled person. A preferred autoclaving temperature would be up to 130° C. In the latter case, only such substrate materials can be used that have a melting point higher than 130° C. For specific applications readily known to the skilled person, higher or lower autoclaving temperatures may be used. Thus, two groups of substrate materials can be distinguished based on their temperature stability, a low- and high-temperature stable group. The low temperature group has a thermal stability of maximally 130° C., the high-temperature group in excess of 130° C. Specific examples of low-temperature stable substrates are LD-PE (melting point: 60-75° C.) and HD-PE (melting point: 90-120° C.). A specific example of a high-temperature stable substrate is PP (melting point: 140° C.).

Chemical Properties:

Substrate materials that are meant to be chemically sterilized must have a chemical stability sufficient to resist such treatment. Specific examples of such materials are LD-PE, HD-PE or PP, all of which have a chemical stability that allows chemical sterilization with typical agents used therefore and known by the skilled person.

Apart from chemical stability that allows sterilization, it is preferable for the present invention that the chemical properties of the substrate are defined and controllable. In particular, it is preferable for the culture substrate of the present invention to be chemically inert. This means, that the substrate should not readily interact with typical ingredients of plant nutrient solutions. In this context, to interact means the compound is adsorbed, precipitated, catalysed, oxidized, reduced, cleaved or chemical groups are added when in contact with the culture substrate. Adsorption in this context means that a physiologically relevant proportion of an added substance adheres to the substrate. This is of particular relevance when the substance is present at low concentration. In a most preferred embodiment of the present invention, the substrate does not readily adsorb lo ingredients of the medium. Typical ingredients of culture media comprise macronutrients such as phosphorus or nitrogen, micronutrients such as various metals, as well as phytohormones. Further, such substances that are typically used in physiological experiments in a scientific setting should not interact with the substrate. These include any xenobiotics such as heavy metals, inorganic compounds, organic compounds, etc., but also comprise amino acids, peptided, proteins, nucleotides, RNA, DNA etc. The list of substances listed here is not exhaustive, but merely represents a selection of examples to illustrate the scope of the chemical space to which the substrate should essentially be chemically inert.

Particular requirements related to chemical inertness are that the culture substrate should not have any free reactive groups, and should not carry a surface charge or exposed charged residues. These properties should be stable over a wide pH range as might be used in culturing plant material. This pH range is from acidic (pH=1) to alkaline (pH=13). Specific examples of materials fulfilling these criteria sufficiently to be useful in the context of the present invention comprise, but are not limited to, PP or PE.

Defined and controllable chemical properties in the context of the present invention may mean for specific applications that chemical reactions, including interactions with the medium or release of compounds are desired.

For example, the granulate can comprise a functional chemical compound, such as a reactive covering. Such covering may contain a pH indicator or a temperature indicator which gives rise to visual information concerning the culturing conditions. Alternatively, the functional chemical compound comprised in the granulate may be any chemical of a defined, desired function, such as chemicals bearing e.g. fungicidal, algaecidal, or microbicidal function. Such chemicals may also comprise substances that function as nutrients or toxins for plant material, or have any other measurable effect on plant material. In some applications, the controlled and sustained release of such compounds will be desirable.

Culturing Vessel

To culture plant material according to the present invention, the culturing substrate will be added to a culturing vessel (the terms vessel and container are used synonymously). The container used for the invention is not limited in size or shape, as the granular substrate will readily form a closed substrate layer independent of container shape or size. Any container from laboratory scale such as glass or plastic tubes, beakers, bowls or troughs up to industrial scale growing chambers or troughs in any desired size, even in the hectar range, can be used. In the case of very large containers, application of the substrate will require appropriate mechanical means.

The combination of cheap and readily available culture substrate and the possibility to use a wide range of culture containers results in a culturing system easily scalable from scientific up to industrial scale. This scalability is independent of expensive investment in e.g. specialised culturing containers and hence provides a significant benefit to the present invention.

Aeration of Culturing Solution

If required, gases such as air or any other gas can be applied to the culturing solution. For this purpose, an opening in the floating substrate layer is introduced. Such an opening can, for example, be introduced by a cork ring with its opening being free of culture substrate. Many other ways of physically creating a substrate free area can readily be envisaged. The desired gas can be applied to the solution by suitable physical means in the area free of substrate. Such means comprise any kind of gas outlet that can be introduced into the solution such that the gas is set free in the region free of substrate.

Culturing Kit

According to one embodiment of the invention, the floatable culture substrate and an appropriate culture vessel form parts of a culture kit. The gist of the present invention is its simplicity, achieved by the self-regulating balance between plant material, substrate and medium. This simplicity is reflected in the culture kit of the invention, which preferably consists of the culturing vessel and the substrate, and optionally, the separately packed culture medium and plant material. One example of the culturing kit is a kit for non-industrial customers, for use for ornamental plants. Accordingly, the culturing vessel will be a decorative vessel, with dimensions as are suitable for growing plants in flats or offices. The vessel containing culturing substrate may also contain seeds of ornamental plants. By adding separately packed culture medium the culturing process can be started.

As an example of a culturing kit for industrial users, stackable culturing containers in a size suitable for stacking on euro-palettes, comprising substrate, with an upper layer comprising seeds of plants, are provided. The containers can be distributed in a green-house, and the culturing process started by simply adding culturing medium.

For shipping the grown plants, excess medium is drained off, and the re-stacked containers comprising the plants are shipped as a whole. Thereafter, containers and substrate can be reused.

Alternatively, the culture kit may just comprise the floatable substrate and separately packed, substrate-seed mixture, etc.

Accordingly, different culturing kits can be specifically adapted to a particular application. As an example, a culture kit may comprise a decorative vessel, containing culturing substrate comprising seeds of ornamental plants, and separately packed culture medium, that simply needs to be added in order to start the culturing process. Alternatively, the culture kit may just comprise the floatable substrate and separately packed substrate-seed mixture, etc. In this way, the present invention provides a diversity of culture kits for hobby, science, or industrial use.

Culturing Process

According to the specific requirements of the plant material to be cultured, any of the following culturing steps may be combined with appropriate measures of sterilization and/or aseptic handling. Sterilization measures for media, containers, substrate, nutrient solution and plant material are widely known in the art. It is obvious to the skilled person which kinds of culturing steps require such measures, therefore they will not be explicitly mentioned in the following description of the culturing method according to the present invention.

Typically, the floatable granular substrate will be added to the culturing container to form a layer of culture substrate. Such a substrate layer may comprise a single type of granules (e.g. HD-PE), or alternatively may comprise a mixture of different types of granules. As an example, the layer may comprise granules made of different materials (e.g. HD-PE granules and PP granules), granules of different elasticity or different optical characteristics. Also, the layer may comprise particles of different sizes.

Plant material may either be added to the granular substrate prior to-layer formation, or thereafter. A sufficient quantity of substrate chosen to support the size and weight of plant material of interest can readily be determined. In this context it is important to note that the substrate layer is floatable, yet, it need not float at all times during the culturing process. It can be envisaged, that at selected time-periods, the substrate layer is either floated or rests on the bottom of the culture container. As an example, a layer of culture substrate may be formed in the culturing vessel prior to addition of culturing medium. Alternatively, the medium may be added first, and culture substrate subsequently.

However, it is emphasized that it is the gist of the culturing method of the present invention that substrate, medium and plant material are in a self-regulated balance that ensures an optimal medium supply to the plant material. Such balanced state is not achieved when the substrate rests on the bottom of the container, or on any other fixed structure, which is not supported by the granule layer itself.

As an example, seedlings may be grown from seeds on a floating layer to allow for optimal nutrient solution supply. Given that the weight of the plant material is relatively small in comparison to the weight of the substrate layer, it will not significantly affect the degree of immersion of the substrate layer. Hence, cleft filling will be optimal for a wide range of plant material weight.

Still, at a later stage, when the seedlings have grown bigger and become too heavy to be supported by the floating layer, the layer may be lowered to the bottom of the container. Then, the seedlings and in particular their roots have developed sufficiently that small variations in solution height within the substrate layer are not as detrimental as at the beginning of the culturing process; However, such a condition does not represent the gist of the invention.

Hence, the culturing method employing a floating layer of culturing substrate is restricted to plant material of appropriate size and weight such to accommodate suspended growth. Obviously, the culturing system may e.g. not be suitable for very large and heavy seeds such as certain nuts, but may easily support fully grown plants of small species.

However, to adapt the method to growing larger plants, additional support structures embedded within and supported by the floatable granular layer may be present. Such structures may e.g. be introduced to increase the mechanical stability of the overall layer, e.g. when culturing particularly large and heavy plant material. In such instances, the physical stability of the layer may need to be enhanced in order to e.g. prevent tilting of large and heavy plants. Such structures may define enclosed spaces within the granulate layer, wherein individual granules are still freely movable. In this context “enclosed” does not mean that physical barriers that substantially restrict medium exchange exist. If a multitude of such structures is present, the spaces between the enclosures is filled by un-enclosed granules. Examples of suitable structures comprise plastic nets with a mesh size smaller than the average granule size, or grids that are interposed in the layer. Bags formed from net material may thus enclose a portion of granules that are part of the layer. In contrast, grids may have considerably larger openings than the average granule size and yet increase the mechanical stability of the layer they are embedded in. In case the embedded structures define enclosures containing portions of particles, such enclosures can be used to separate the plant material attached to them from the rest of the layer, e.g. for harvesting and 1 or replanting.

Alternatively, the substrate layer may contain agglomerates or aggregates of substrate particles of different sizes, wherein individual particles are attached to other particles to a variable degree and with variable attaching strength. Such structures would be interspersed with unattached particles to form a dense substrate layer.

Importantly, none of the additional structures or particle aggregates must impede the basic principle of the invention, namely the self regulated balance between medium, substrate and plant material.

The thickness of the layer of the floatable granular substrate is not limited, as long as the layer provides sufficient stability and buoyancy for the chosen application. Obviously, the thickness should be adjusted to the size and weight of the plant material to be cultured. The thickness can be adjusted either at the beginning of the culturing process by adding the appropriate amount of substrate material to the culturing vessel, or can be adjusted during the culturing process e.g. by adding substrate particles from below in a suitable culturing vessel. Hence, the present invention allows the continuous adaptation of the substrate layer thickness to optimal culturing conditions without any disruption of the plant material.

In a preferred application growing plant material such as seedlings, meristems or calluses, the thickness of the substrate layer will be in the range of 0.5-20 centimetres, preferably 0.5-15 centimetres, preferably 1-10 centimetres, more preferably 2-8 centimetres. Apart from the thickness of the layer, the average size of the culture substrate granules can be adjusted to the size and weight of the plant material to be cultured.

When using small seeds, such as e.g. derived from Arabidopsis or Thlaspi, an average granule size of 1-2 millimetres and a layer thickness of 2-3 centimetres are recommended. For larger seeds, such as e.g. barley, an average granule size of up to five millimetres and a layer thickness of approximately 5 centimetres is suitable. For even larger or heavier plant material, such as large seeds, calluses or meristems derived from tobacco, lettuce, or tomatoes, layer thickness may be increased up to approximately 8 centimetres obviously, culturing of very large plant material will require layer heights in excess of 8 centimetres. There is no theoretical upper limit of layer thickness, other than the restrictions imposed by the culturing vessel.

When the granular substrate has been distributed in the culturing container to the desired layer thickness, plant material may simply be placed on top of the substrate layer or can be stuck into the layer to a desired depth.

An alternative to placing plant material on top of the substrate layer is mixing it with substrate. This is a preferred method when e.g. culturing seedlings from seeds. The seeds can be mixed with an excess of culture substrate and the resulting mixture can be distributed in a thin layer on top of the substrate layer. The seeding density can readily be adjusted by choosing different ratios of seeds/culture substrate. A typical ratio would involve e.g. approximately a ten-fold volume of culture substrate compared to seeds.

The simple preparation of the substrate layer by distribution in an appropriate culture container, in connection with the easy application of plant material represents a significant advantage over many conventional systems. Hence, laborious substrate preparation such as e.g. in an agar-based system, floating sponges etc. is not required. Also, the often most laborious step, the correct positioning of plant material, is greatly facilitated. This is particularly obvious in comparison to conventional floating systems, where plants need to be placed in or on top of individual openings. It can be envisaged, that the present system will allow machine-planting in an industrial setting, where at present plants are positioned by hand.

Any appropriate culture medium can be used for the present invention, given that the density of the granular substrate is chosen accordingly. A wide variety of different nutrient solutions are in routine use in hydroponic plant culture. Examples of such solutions comprise, but are not limited to, Hoagland's solution or MS-basal medium.

The nutrient solution is either added by pouring it in at an edge of the container, or it can be flooded from the bottom of the container, depending on the technical specification of the culturing container obviously, culturing vessels can also be constructed such that a permanent or intermittent flow of medium through the vessel and the substrate layer therein occurs. Such flow can be used to achieve defined culturing conditions at a given point in time, e.g. the temporally defined addition or withdrawal of certain substances, or the complete exchange of medium. Such flow may also contribute to providing sufficient medium of a precisely defined composition and physical parameters to the plants.

In a particular application, flow through systems can be employed e.g. to establish an online indicator system in e.g. a waste water stream, wherein a readily detectable change in the culturing system would indicate a defined environmental condition. Such flow through may also be employed when using the present invention in test systems other than online sensors.

When medium is added, initially, the clefts of the culturing layer will fill with solution. Then, if more solution is added, the substrate layer will begin to float. The height of the solution phase under the substrate layer depends on the amount of solution added. The height of the solution phase can freely be chosen according to the specifications of the container and the desired culturing conditions.

For some applications, such as e.g. further culturing of seedlings or plants that already have a developed root system, it may be advisable to plant on a floating substrate layer, rather than before nutrient solution is added. Of course, all other plant material can also be added at this point rather than before solution is added.

Now, plant material will grow in the hydroponic system. Roots will find both sufficient physical stability to support growth, and the necessary tactile stimuli for development of primary roots. In case the plant material develops roots and is grown for a sufficient length of time, and given that the culture substrate is floating, the roots may grow out of the substrate layer into the nutrient solution.

Hence, an advantage over e.g. available membrane based culturing systems is that root development is not restricted by any solution/membrane interfaces.

If the culture substrate is floating, the excess of nutrient solution will provide sufficient water and nutrients for the plant material over a prolonged period of time, and will dissipate toxic exudates. This represents an advantage over all systems that contain areas of restricted nutrient solution volume, such as commercially available membrane based floating systems. Further, the solution-air interface will remain unchanged at the upper edge of the substrate layer for a wide range of solution volumes. This is achieved by self-regulation, without further regulating devices. This is an advantage over conventional, non-floating systems.

If necessary, the culturing solution can readily be exchanged during the culturing process, without physically disrupting the plant material. Thus, for example, different nutrient solutions can be used for different growth phases of the plant material. Such an exchange is impossible in soil-grown plants, but also in e.g. agar-based systems. Also, hydroponic systems employing e.g. expanded clay may exhibit a substantial retention effect due to the extensive inner surfaces of that substrate.

To harvest plant material, it can simply be pulled out of the substrate layer, without causing any physical disruption to the plant material. This is a requirement both in industrial applications, where the plants are cultivated further, and in science, when intact plant material needs to be harvested. In comparison, if plants grow through membranes, they need to be carefully excised. Also, any systems employing rock wool or agar make it very difficult to separate roots from substrate.

Further, such systems do not allow to readily replant in a different culturing container, in particular where a root system has developed. Non-floating hydroponic systems require emptying of the substrate. Agar-based systems require excision of individual plants. The remaining agar may produce rot if transferred onto e.g. soil as a substrate for further growth. Systems where plants grow through holes in a supporting structure can result in mechanical damage to plant material, or the material may even get stuck when growing for too long.

Consequently, all these systems are labour intensive in replanting, and some do not allow replanting of root bearing plants at an industrial scale at all. In contrast, when using a floating, granular substrate layer, plants can easily be harvested and easily be replanted. For replanting, the plants can simply be gently pushed through the layer of floatable substrate, until the roots can spread freely in the solution phase, and the body of the plant is firmly anchored in the substrate.

Thus, the floatable granular substrate of the present invention may facilitate automated harvesting and/or replanting devices that cannot readily be used on conventional systems.

Further, it is possible to add a layer of conventional substrate like soil at a given point in the culturing process, to allow the plant material to grow into said conventional substrate. Such a process may e.g. facilitate and speed up consecutive replanting and hardening.

Previously, a culturing process employing PE beads has been described (Hart J J et al., 1998 a and b). In this process, seeds were germinated on filter paper. Then, seedlings were placed on mesh bottom cups and covered with black PE beads to shield them from light. The cups were suspended in a vessel containing medium. Thus, the PE beads in this process are not a culturing substrate in the sense of the present invention, as they do not provide sufficient support from underneath to prevent the plant material from sinking. Rather, the plant material is supported by the mesh bottom of the culturing cups. The beads are functioning as a mere cover that weighs the seedlings down on the mesh bottom and shields them from light. Further, the PE beads are not a culturing substrate in the sense of the present invention as they do not allow germination of seeds and culturing seedlings without repositioning. Also, the beads are not meant to float, quite the contrary, the beads were used as weights to firmly position the seedlings on the mesh bottom. Finally, in this culturing process the mesh bottom forms a mechanical structure that permanently separates plastic beads and free solution phase throughout the culturing period. The culturing process of the present invention does not require such a mechanical barrier.

### EXAMPLES

Culture Medium

MS-basal medium (Sigma) was preferably used for culturing plant material in the consecutive examples. Alternatively, Hoagland's E-Medium was prepared as follows (additions per liter of final medium):

| | Stock solution | Addition to final medium |
|---|---|---|
| Substance | (g/100 ml) | (ml/l) |
| MgSO₄.7 H₂O | 24.6 | 1.0 |
| Ca(NO₃)₂.4 H₂O | 23.6 | 2.3 |
| KH₂PO₄ | 13.6 | 0.5 |
| KNO₃ | 10.1 | 2.5 |

| | |
|---|---|
| Micronutrients (see below) | 0.5 |
| Fe.EDTA Solution added last (see below) | 20.0 |

Adjust pH to 5.8 with NaOH or HCl. Sucrose may be added as 10 g/l if the culture is axenic. Autoclave.

Micronutrient Stock Solution:

| | |
|---|---|
| H₃BO₃ | 2.86 g/l |
| MnCl₂.4 H₂O | 1.82 g/l |
| ZnSO₄.7 H₂O | 0.22 g/l |
| Na₂MoO.4 H₂O | 0.09 g/l |
| CuSO₄.5 H₂O | 0.09 g/l |

Fe.EDTA Stock Solution:

| | |
|---|---|
| FeCl₃.6 H₂O | 0.121 g/250 ml |
| EDTA | 0.375 g/250 ml |

Dissolve completely and make up to 250 ml. After autoclaving medium, add Fe•EDTA Stock Solution aseptically.

Culturing Conditions

In the examples the following growing conditions were used unless specified otherwise: Plant material was cultivated for 6 d to 14 d in a controlled environment cabinet (relative humidity 70+/−5%) under a 14/10 h day-night cycle with a photosynthetic active radiation of 116 82 M·m⁻² s⁻¹ from 06:00 to 20:00 h middle European summer time and a temperature of 24/20° C.

### Example 1

### Culturing Barley on Floating Culture Substrate

Plant Material:

Hordeum vulgare cv. Barke was used. Cv. Barke (BSA-Nr. 1582) is an awned, double-lined summer barley, which were breeded 1996 by Breun from Libelle X Alexis.

Pre-Treatment of Barley Caryopses Under Axenic Conditions:

Caryopses were incubated for 1 min in ethanol (70 vol/vol %) and then for 1 min in H₂O distilled. Caryopses were then incubated two-times for 5 min in NaOCl (10 vol/vol % of a stock solution containing 6-14% free, active chloride), containing 0.1 vol/vol % Triton X-100. The caryposes were then washed ten-times for 1 min with H₂0 distilled and swelled for 24 h in H₂O distilled at room temperature.

Floatable Substrate Layer

LD-PE-granulate with an average granule size of 3-5 millimeters was used. A layer of granulate of approximately 4 cm in height was poured into different glass beakers. The beakers ranged in size from 50-5000 ml, with a corresponding diameter of approximately 4-25 centimetres. Alternatively, rectangular polyethylene troughs with a size of approximately 50×70 centimetres and a height of approximately 10 centimetres were used. The required volume of granular substrate was 40 l/m². The caryopses from Example 1 were mixed with PE-granulate (1:10; v/v). This mixture was distributed on top of the PE-granulate layer, resulting in a seedling density of 10-15 plants per 10 cm². Alternatively caryopses were applied directly onto the PE-granulate.

Then, Hoagland's medium or MS-basal medium was added by pouring into the culture container at the edge. Pouring speed was chosen such as not to upset the substrate layer. The solution volume was chosen such that a liquid phase of approximately 4 cm was formed underneath the floating substrate layer.

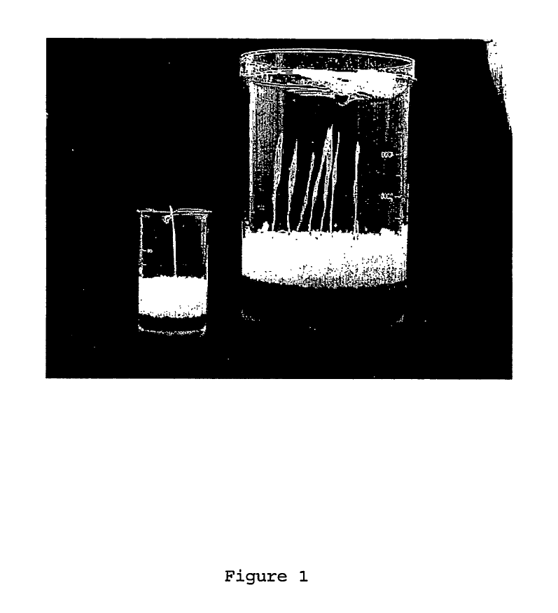

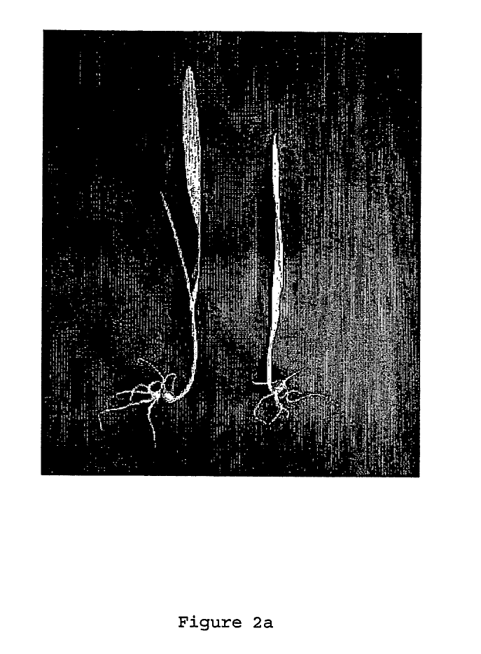

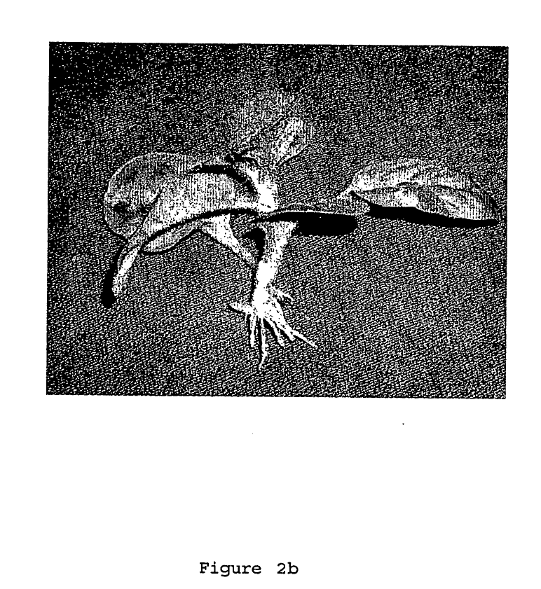

Seedlings were grown as indicated. Thereafter, seedlings were harvested by gently pulling them out of the substrate layer. Representative seedlings obtained by this method are depicted in FIGS. 1 and 2a. The seedlings had reached an average height of 100-150 mm and an average weight of up to 2 g, and showed a normal morphology. These values correspond well with such known from the literature for respective seedlings grown on e.g. agar substrate.

Hence, floatable substrate is capable of supporting normal growth of barley seedlings in large quantities under strictly controlled culture conditions.

### Example 2

### Comparison of Heavy Metal Uptake of Barley Seedlings on Floating Culture Substrate or Agar Substrate

MS-basal Medium or Hoagland's medium was supplemented with heavy metals as follows: stock solutions of Hg Cl₂ (0.1 M) or Cd(NO₃)₂•4H₂O (0.1 M) were applied to the autoclaved medium when it had cooled to about 55° C. such that final heavy metal-ion concentrations of 10, 20, 30 and 40 μM were obtained.

Agar culturing substrate was prepared by adding 1.5 wt/vol % agar (Sigma) to Hoagland's or MS-basal medium. After heating to 100° C., respective amounts of heavy metal salts were added when the medium had cooled down to 550° C. Then the mixture was poured into Phytotray II™ culture vessels (Sigma) and left to cool for subsequent use.

Barley caryopses prepared and seeded onto a floatable substrate layer as in Example 1. Alternatively, the caryopses were directly placed on culturing agar as described above.

Seedlings were grown as described, and leaves and roots of 6 day old plants were harvested and weighed to determine the fresh weight. Samples were shock-frozen in liquid nitrogen and stored at −80° C.

Total mercury and cadmium content of leaves and roots was determined by electrothermal vaporization inductively coupled plasma-mass spectrometry (Michalke et al., 1997).

The results are shown in Table 1:

TABLE 1
Concentration of heavy metal ions (μM) in barley
seedlings grown on agar or on floatable culture substrate.

| | | |
|---|---|---|
| added | Agar | Floatable Substrate |

| concentration | | Shoot | Shoot | Root |
|---|---|---|---|---|
| Hg²⁺ | 0 | 2 | 1 | 54 |
| | 10 | 62 | 54 | 697 |
| | 20 | 171 | 144 | 851 |
| | 30 | 254 | 239 | 2060 |
| | 40 | 252 | 296 | 2430 |
| Cd²⁺ | 0 | 2 | 15 | 55 |
| | 10 | 15 | 4 | 479 |
| | 20 | 21 | 27 | 1270 |
| | 30 | 41 | 27 | 1450 |
| | 40 | 53 | 59 | 2880 |

The results show that uptake of Hg²⁺ and Cd²⁺ in barley shoots grown on floatable substrate or on agar medium is comparable. As expected there was a positive correlation between the metal-ion concentration in the medium and the concentration in the shoot tissue. The concentration of Hg²⁺ in the roots of seedlings grown on floatable substrate was increased by a factor of 10 compared to shoot tissue, and by a factor of 60 for Cd²⁺.

Importantly, metal ion concentrations could not be established for root material grown on agar, because the roots could not be separated from the substrate material. Hence, metal ions contained in the substrate would have been measured in addition to those contained in the root material.

### Example 3

### Growing Arabidopsis or Thlaspi Seedlings

A floatable substrate layer was prepared as described. Yet, particle size of the granulate was selected to comprise 1-5 mm, and a layer height of 3 cm was chosen. After adding MS-basal medium, seeds were directly applied to the upper surface of the layer. The seeds were germinated and cultivated for 21 days under conditions as described. At 21 days, the plants were harvested and placed on a dry filter paper to absorb attaching culture solution. The plants had reached a fresh weight of 0.5-2 g.

### Example 4

### Growing Calluses on Floatable Culture Substrate

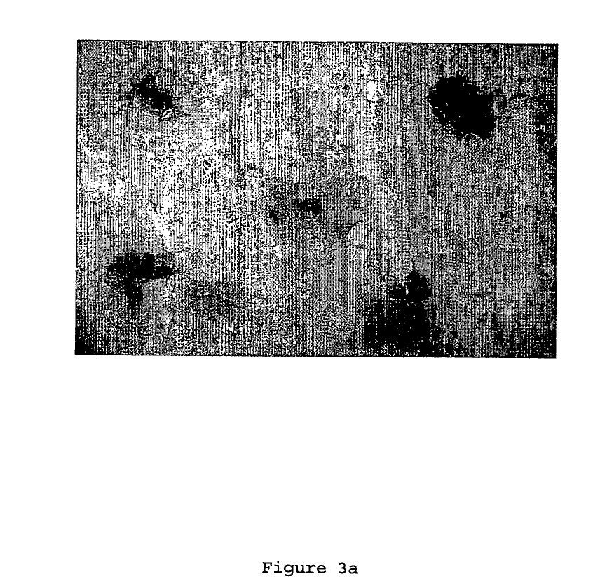

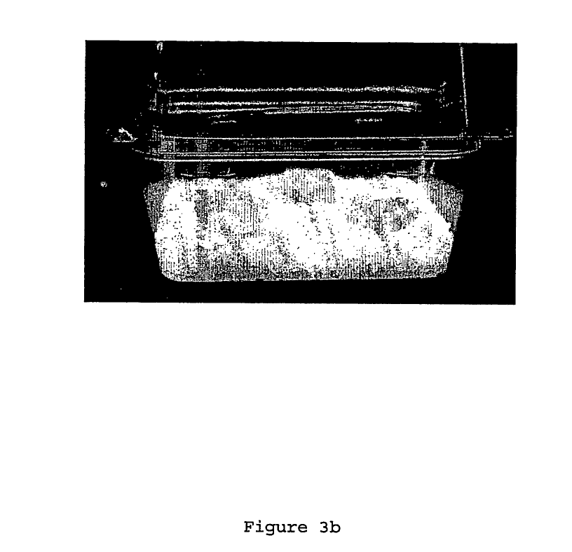

Tobacco internodes were surface sterilized as described. Floatable culture substrate (5-8 mm) and a MS-basal medium were filled into a glass culturing container such that a layer of, approximately 3 cm height with a solution phase of equal height were formed. The glass container was lidded and autoclaved. After cooling, an internodial section of approximately 1 cm length was positioned on top of the substrate layer under sterile conditions. An appropriate amount of sterile IES and NAA stock solutions known to be useful for dedifferentiating tobacco tissue was added. The lid was sealed and the container placed in a controlled environment cabinet (relative humidity 70+/−5%, 24° C.) and cultivated in the dark. After two weeks, callus tissue was separated from the internodal section under sterile conditions, was mechanically dissociated into smaller pieces and was placed in a new culturing container prepared as described. A representative picture of callus obtained after an additional week of culturing is shown in FIG. 3a, callus obtained after additional culturing for four weeks is shown in FIG. 3b.

### Example 5

### Growing Meristems Derived from Tobacco Plants on Floatable Culture Substrate

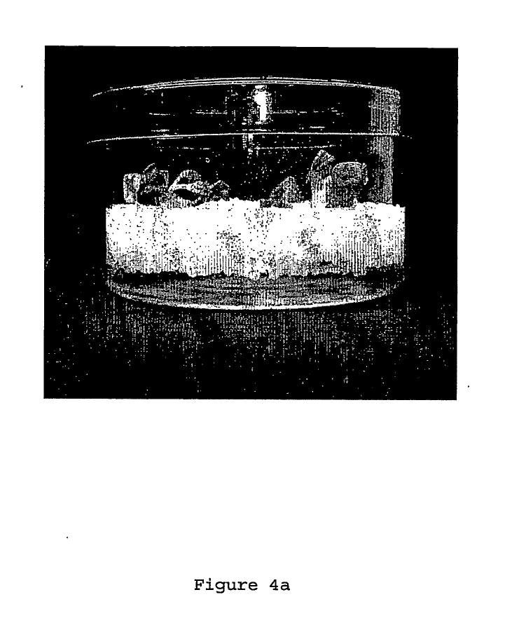

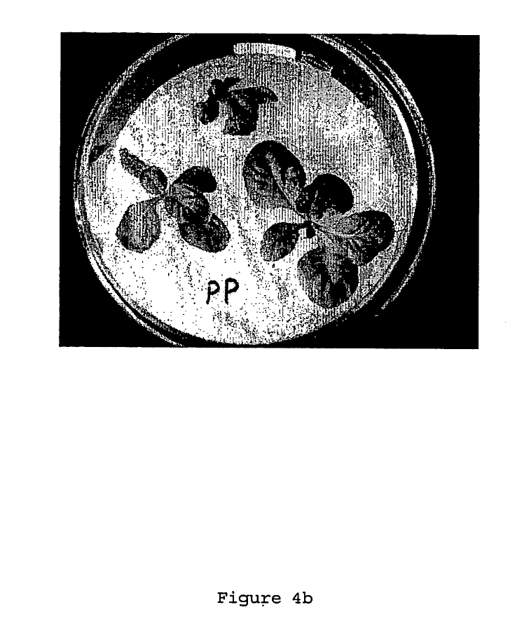

Tobacco plant shoots were harvested and surface sterilized as described. Under sterile conditions, the top two to three internodes of each shoot were dissected such that 6-8 leaves were preserved. The plant material had a height of approximately 30 mm and weighed approximately 5-7 g. Floatable culture substrate (5-10 mm) and a MS-basal medium were filled into a glass culturing container such that a layer of approximately 3 cm height with a free solution phase of approximately 1 cm height were formed. The glass container was lidded and autoclaved. After cooling, the tobacco material was gently pushed into the substrate layer, taking care that none of the leaves was fully submerged (FIG. 4a). An appropriate amount of sterile IBA stock solution known to be useful for promoting root growth on tobacco tissue was added. After 10 days of culturing, the tobacco meristems showed richly developed primary roots and began to grow secondary roots (FIG. 2b). The plants were supported by the substrate in an upright position, with notable growth of leave tissue (compare FIGS. 4a and 4b).

### Example 6

### Growing Tobacco Plants

A floatable culture layer with a height of 8 cm was prepared in a rectangular PE culturing vessel with a volume of 50 1. After adding approximately 20 1 of MS basal medium to form a floating layer, the tobacco meristems of Example 5 were replanted in said layer at approximately 2 plants per 10 cm². The meristems carrying rich primary roots were pushed into the substrate layer to a depth of approximately 2-2.5 cm, taking care that none of the leaves was fully submerged. The plants were grown under conditions as described for two weeks. The plants were harvested by gently pulling them out of the substrate layer. Adhering substrate material was detached by gently shaking the plants. At this point, the plants had reached an overall size of approximately 8-12 cm and a weight of 14-20 g. The shoots had grown to approximately 4-6 cm and had a normal morphology. The plants had a richly developed primary and secondary root system. The plants were replanted in conventional soil to be transferred to a greenhouse for hardening.

### Example 7

### Determination of the ‘No Adverse Effect Level (NOAEL)’ for Xenobiotics According to OECD Guideline 208

The plant of interest is selected. Amongst crop species e.g. the following can be used: tomato, cucumber, lettuce, soybean, cabbage, carrot, perennial ryegrass, corn or onion. A floatable culture substrate layer of appropriate granule size and height (preferably 3-8 cm) for the species of interest, is added to a culture vessel with a surface area of 15 cm². MS basal medium or Hoaglands medium is prepared as described and added to the culture vessel such that a free solution phase of ⅓ to 1×the height of the substrate layer is present. If appropriate, the vessel is lidded, autoclaved and left to cool prior to use.

The plant material is added at a plant density as follows: One or tow corn, soybean, tomato, cucumber or sugar beet plants per 15 cm container, three rape or pea plants per 15 cm container, 5-10 onion, wheat or other small seeds per 15 cm container. The number of containers is chosen to accommodate the planned range of different concentrations and replicate pots for each concentration of xenobiotic. A minimum of 20 plants per concentration, divided into a minimum of four replicates is required.

Stock solutions of the xenobiotic of interest are prepared and sterile-filtered, or autoclaved if appropriate. Appropriate amounts of the stock solution are added to each of the containers to prepare a concentration range of the test substance (e.g. 0.1, 1.0, 10, 100 and 1000 mg/l medium). The test series also includes reference pots containing no test substance, as well as at least one concentration of a different test substance with a known effect on the plant of interest. Solvent controls may be required for xenobiotics not dissolved in water. In every other respect, the control containers will be treated identical to the test containers. The test conditions should approximate those conditions necessary for normal growth for the species and varieties tested. To allow for defined culturing conditions, a growing chamber, phytotron, greenhouse etc. can be used. For the listed species the following conditions are recommended: carbon dioxide concentrations: 350+/−50 ppm, relative humidity: 70+/−5% during light periods and 90+/−5% during dark periods, temperature 25+/−3° C. during the day, 20+/−3° C. during the night, photoperiod: 16 h light/8 h darkness, assuming an average wavelength of 400 to 700 nm, light: luminance of 350+/−50 micromol/m²/s, measured at the top of the canopy.

The plants are cultured for an observation period of 14-21 days after 50% of the control plants (also possible solvent controls) have emerged, the plants are observed frequently (at least weekly) for visual phytotoxicity and mortality. At the end of the test, measurement of % emergence and biomass should be recorded as well as visual phytotoxicity (chlorosis, necrosis, wilting, leaf and stem deformation). For evaluation the plants are harvested by pulling them out of the substrate layer, gently shaking off adhering substrate particles and briefly rinsing with destined water. Both the shoot and the root system can be evaluated. Regarding the root system, primary, and secondary roots as well as root hairs can be evaluated. Biomass can be measured using final shoot and root weight, preferably dry weight by harvesting and drying at 60° C. to a constant weight. The results are recorded and evaluated using standard statistical procedures to calculate an EC₅₀, NOAEL etc.

### REFERENCES

Arteca R N and Arteca J M (2000) A novel method for growing Arabidopsis thaliana plants hydroponically. Physiol Plant 108: 188-193.

Feng J, Zheng S J, Li X F, Takeda K and Matsumoto H (1997) A rapid hydroponic screening for aluminium tolerance in barley. Plant Soil 191: 133-137.

Gibeaut D M, Hulett J, Cramer G R and Seemann J R (1997) Maximal biomass of Arabidopsis thaliana using a simple, low-maintenance hydroponic method and favourable environmental conditions. Plant Physiol 115: 317-319.

Hart J J, Welch R M, Norvell W A, Sullivan L A, Kochian L V (1998) Characterization of Cadmium Binding, Uptake, and Translocation in Intact Seedlings of Bread and Durum Wheat Cultivars. Plant Physiol. 116: 1413-1420.

Hart J J, Norvell W A, Welch R M, Sullivan L A, Kochian L V (1998) Characterization of Zinc Uptake, Binding and Translocation in Intact Seedlings of Bread and Durum Wheat Cultivars. Plant Physiol. 118: 219-226.

Heidenreich B (1999) Transkriptionsänderungen von Arabidopsis thaliana bei Ozon-, UV-B und HgCl2-Behandlung. PhD thesis, LMU München.

Huttner D and Bar-Zvi D (2003) An improved, simple, hydroponic method for growing Arabidopsis thaliana. Plant Mol Biol Reptr 21: 59-63.

Leal R S (2001) The use of Confidor S in the float, a new tobacco seedlings production system in the south of Brazil. Pflanzenschutz-Nachrichten Bayer 54, 3, 337-352.

Michalke B, Lustig S and Schramel P (1997) Analysis for the stability of platinum-containing species in soil samples using electrophoresis interfaced on-line with inductively coupled plasma mass spectrometry. Electrophoresis 18: 196-201.

Thiel S, Döhring T, Köfferlein M, Kosak A, Martin P and Seidlitz H K (1996) A phytotron for plant stress research: How far can artificial lighting compare to natural sunlight? J Plant Physiol 148: 456-463.

Tocquin P, Corbesier L, Havelange A, Pieltain A, Kurtem E, Bernier G and Përilleux C (2003) A novel high efficiency, low maintenance, hydroponic system for synchronous growth and flowering of Arabidopsis thaliana. BMC Plant Biol 3: 2.

1.-35. (canceled)

36. A method for culturing plant material comprising the steps of:
(a) forming a layer of floatable granular substrate in a culturing vessel,
(b) placing plant material on or in said layer, and
(c) culturing the plant material in the presence of a culture medium, wherein there is no additional structure supporting the plant material from underneath,
wherein said floatable granular substrate comprises particles having an
average diameter of 1-25 mm.

37. The method according to claim 36 wherein the culture medium is added before the layer of the granular substrate is formed.

38. The method according to claim 36 wherein the culture medium is added after the layer of the granular substrate is formed.

39. The method according to claim 36 wherein said particles have an irregular polygonal or spheroidal shape.

40. The method according to claim 36 wherein said particles have a regular polygonal or spheroidal shape.

41. The method according to claim 36 wherein said granular substrate comprises particles having a smooth surface.

42. The method according to claim 36 wherein said granular substrate is chemically inert.

43. The method according to claim 36 wherein said granular substrate is a thermoplastic polymer.

44. The method according to claim 43 wherein the thermoplastic polymer is selected from the group consisting of HD-PE, LD-PE and PP.

45. The method according to claim 36 wherein the granular substrate has a density of 0.90-0.96 g/cm³.

46. The method according to claim 36 wherein said particles comprise at least one hollow enclosure.

47. The method according to claim 36 further comprising the step of sterilizing the granular substrate by chemical treatment, irradiation or heat.

48. The method according to claim 36 wherein the granular substrate forms a substrate layer and wherein said substrate layer is 0.5-20 cm thick.

49. The method according to claim 48 wherein said substrate layer floats on the culture medium.

50. The method according to claim 49 further comprising the step of aerating the culture medium.

51. The method according to claim 48 wherein said substrate layer comprises additional embedded support structures, wherein said additional support structures are supported by the granular substrate layer.

52. A culturing kit for culturing plant material comprising a culturing solution, a granular culture substrate floatable in the culturing solution, and a culturing vessel, wherein the granular culture substrate comprises particles having an average diameter of 1-25 mm.

53. The kit according to claim 52 wherein the granular substrate is chemically inert.

54. The kit according to claim 53 wherein the granular substrate is a thermoplastic polymer selected from the group consisting of HD-PE, LD-PE and PP.

55. Use of a floatable granular substrate for culturing plant material, wherein the granular substrate is comprised of particles having an average diameter of 1-25 mm, and wherein the granular substrate has a density of 0.5-1.1 g/cm³.
